(19) United States
(12) Patent Application Publication
(10) Pub. No.: US 2014/0087331 A1
(43) Pub. Date: Mar. 27, 2014

(54) DENTAL IMPLANT HAVING A FIRST, CONICAL, SCREW PART AND A SECOND, CYLINDRICAL, SCREW PART

(71) Applicant: Riemser Pharma GmbH, Greifswald-Insel Riems (DE)

(72) Inventors: Martin Hildmann, Bad Homburg (DE); Stefan Naegele, Neu-Ulm (DE)

(21) Appl. No.: 14/033,650

(22) Filed: Sep. 23, 2013

Related U.S. Application Data
(63) Continuation of application No. PCT/DE2012/100072, filed on Mar. 22, 2012.

(30) Foreign Application Priority Data
Mar. 22, 2011 (DE) 10 2011 001 485.3
May 27, 2011 (DE) 10 2011 050 678.0

(51) Int. Cl. A61C 8/00; A61K 6/04

(52) U.S. Cl. CPC A61C 8/0025; A61K 6/04; A61C 8/0024; A61C 8/0001; A61C 8/006
USPC 433/174

(57) ABSTRACT

The invention relates to a dental implant having a first conical screw part and a second cylindrical screw part, wherein the first screw part has a progressive thread and the second screw part has a fine thread and there is a circumferential groove at the thread termination of the fine thread, wherein the implant has a hexagon socket at the cervical end, the hexagon socket being arranged in a conical recess.

### CROSS-REFERENCE TO RELATED APPLICATIONS

This is a continuation of International patent application no. PCT/DE2012/100072, filed Mar. 22, 2012 designating the United States and claiming priority to German Patent Application DE102011001485.3, file Mar. 22, 2011 as well as to German Patent Application DE102011050 678.0, file May 27, 2011. All of these applications are incorporated herein by reference in their entirety,

### FIELD OF THE INVENTION

The invention relates to a dental implant having a first screw part that is conical and a second screw part that is cylindrical, the first screw part having a progressive thread and the second screw part having a fine thread and a peripheral groove being provided at the thread end of the fine thread. In addition, the invention relates to a kit comprising a dental implant.

### BACKGROUND OF THE INVENTION

Dental implants are artificial dental roots that are used for cases in which the tooth and/or the root is/are damaged or diseased. These implants have a helical or cylindrical design and are implanted in the jawbone to replace lost teeth. The implant in principle assumes the same function as the patient's own dental roots because it fuses with the bone. In the subsequent course of a treatment, crowns or bridges constituting a dental prosthesis may be positioned using the implant.

In the superstructure of a dental implant, a distinction is made between the implant body, which is inserted into the bone, and the superstructure parts, with the help of which the crowns or retaining structures for prostheses are attached to the implant body. A distinction is made here between one-piece or multi-piece implants, in which the implant body and the superstructure part are separated. However, there are also implant systems in which the connecting element to the dental prosthesis (stump for crowns, ball-head anchors for fixation of prostheses) are processed in one piece with the implant body.

Dental implants have been used in dentistry for approximately 40 years; and over the years, various materials have been found to be especially advantageous for fabrication of these implants. Thus, for example, implants made of ceramic or metal have been used. The part of the implant inserted into the tissue is made of high-purity titanium because its surface-passivating oxide layer consisting of titanium dioxide is especially biocompatible and forms a secure bond with the bone. Optimal osseointegration can be ensured in this way. Implants made of titanium are biologically neutral and also do not cause any allergic or foreign body reactions in the throat or oral cavity.

The prostheses for implants or the implants themselves may also be made of ceramic, but zirconium oxide ceramic is the preferred material in the present case. This ceramic implant or a ceramic prosthesis is made of high-strength zirconium oxide ceramic, which has extremely high fracture strength and, according to experience available so far, is also highly biocompatible. In addition, it has the advantage that it approximates the natural tooth color (white instead of gray) much more closely, which may be advantageous in cosmetic dentistry in particular.

The publications and other materials, including patents and patent application, used herein to illustrate the invention and, in particular, to provide additional details respecting the practice are incorporated by reference in their entirety.

Numerous implant structures have been described in the prior art. For example, DE 10 2009 027 044 A1 discloses a multipart dental implant having two shaft parts, one of which is designed as a superstructure shaft part and the other as a root shaft part. The shaft parts are joined together by an implant-abutment connection, which is designed so that the one shaft part has an axial protrusion engaging in the corresponding axial recess in the other shaft part.

In addition, WO 2007 031 562 (see also US 2009/0123889) describes a multipart dental implant, which ensures, on the basis of the geometry of the connection between the root shaft part and the superstructure shaft part, that the connection of the two elements is twist-proof. It is ensured that the surfaces of the root shaft part and the superstructure shaft part contacting one another ensure a defined distance between opposing sealing surfaces, causing a defined compression of a sealing element arranged between the sealing surfaces.

The abutment (also referred to as the superstructure) of a dental implant is applied to the implant by means of a retaining screw. Inclined positions of the implants implanted in the jawbone can be compensated by means of the abutments. However, it is necessary to ensure here that the abutment is designed to be twist-proof in relation to the implant in order to prevent twisting of the abutment and the implant when tightening or loosening the abutment screw (retaining screw). The abutment screw (retaining screw) should also be seated securely because otherwise the superstructure may be loosened. Abutments are usually screwed into the implant by means of abutment screws and are tightened with a defined torque using a torque wrench. The screwdriver tools used for assistance and the abutment screw are conventional hexagon screwdrivers or flat-tip screwdrivers. Implants and abutments that are present in the form of a joint component or as a separate unit are described in the prior art. Such implant systems are described in DE 10 2006 005 66 A1, DE 19803172 A1, DE 10 2006 005 147 A1 or EP 0801544 A1, for example.

In addition, US 2004/0101808 A1 describes an implant system having a double anti-rotational design between the implant and the superstructure, such that the implant has a chronic [sic; conical] shape with a rounded apical end. U.S. Pat. No. 6,402,515 W1 describes an implant having a chronic [sic; coronal] fastening part and a simple progressive thread by means of which fusion of the dental implant in the bone is to be improved. In addition, US 2004/0219488 A1 describes a so-called micro-thread which follows from a two-start thread. This micro-thread contains a number of small helical grooves.

WO 2004/098442 discloses a screw-in dental implant, in which groove-like recesses running in the direction of the pitch of the screw thread overlap with a relatively coarse thread and form a micro-thread to increase the contact area between the dental implant and the bone tissue. The threaded pin itself here is essentially cylindrical and the thread is not designed to be progressive.

One disadvantage of the dental implants disclosed in the prior art is that they do not form a reliable barrier for bacteria, so there are various possible infections of the hole and/or the jawbone or the oral cavity. Furthermore, the fusion time of the implants may be very long (single-stage or two-stage approach), so that a patient must expect massive restrictions in his quality of life for a long period of time.

Accordingly, one of the needs on which the present invention was based was to provide a dental implant that would not have certain disadvantages or shortcomings of the prior art. In one embodiment, the present invention is directed at addressing these and other needs.

### BRIEF SUMMARY OF THE INVENTION

In one embodiment, the invention relates to a dental implant having a first screw part that is preferably minimally conical and a second screw part that is cylindrical; the first screw part may have a progressive thread which may become progressively wider, starting from the rounded apical end, and the second screw part may have a fine thread connected to the progressive thread. There is also preferably a peripheral groove on the thread end of the fine thread, so that the implant has a hexagon socket head on the cervical end arranged in a conical recess.

For example, a first screw part (e.g., 3) is conical, and a second screw part (e.g., 5) is cylindrical. The first screw part may have a progressive thread (e.g., 3A), which becomes progressively wider starting from the rounded apical end (e.g., 2), and the second screw part may have a fine thread (e.g., 5A) connected to the progressive thread. There may be a peripheral groove (e.g., 6) at the thread end of the fine thread, wherein the implant (e.g., 1, which may, e.g., be made out of titanium and/or may have an etched and/or sandblasted surface and/or may have a bore (e.g., 10)) may have a hexagon socket head (e.g., 8) on the cervical end (e.g., 4) arranged in a conical recess (e.g., 9).

It was completely surprising that a dental implant could be provided that does not have the disadvantages of the prior art and permits a long-lasting integration into a jawbone. The first screw part is preferably designed to be minimally conical, so that the conical portion is limited to a minimum to thereby imitate the shape of a dental root which in turn greatly simplifies the introduction of the implant. The implant may preferably be designed with different lengths, in particular 3 mm to 20 mm, preferably 5 mm to 18 mm, in particular preferably 5 mm to 15 mm. The diameter of the implant may also be designed variously, diameters from 1 mm to 10 mm, preferably 2 mm to 8 mm, especially preferably 3 mm to 6 mm being advantageous. It was also completely surprising that an implant could be provided that is easily screwed into the jawbone.

The implant preferably serves to receive and may receive the dental prosthesis for example, via which the chewing function can be restored. To permit optimal attachment of the implant in a bone pocket in the jawbone, the implant may be provided with a thread, which is progressive on the apical end. The progressive thread is preferably a multi-start thread (e.g., a two-start self-cutting thread) which becomes wider from the apical end to the cervical end, such that the progressive thread is connected to a micro-thread or to a fine thread. The fine thread may also be embodied as a multi-start thread (e.g., a triple-start thread).

A progressive thread already known from U.S. Pat. No. 6,402,515 becomes progressively wider from the rounded apical end of the implant and significantly improves the fusion of the dental implant in the bone. With a thread depth that increases continuously in the apical direction in particular, the application of load is shifted into the more yielding spongy bone substance. Furthermore, it has been found that the bottom profile of the threads also guides a lateral force perfectly into the bone and thus contributes toward optimal relief for the cortical bone. The first progressive screw part, which is designed with a conical shape, preferably has a conicity of 0.001° to 10°. The length of the conical first screw part is preferably 1 mm to 10 mm, preferably 2.5 mm to 7.5 mm, especially preferably 4 mm to 5 mm.

Due to these technical designs, it is possible to cover all therapeutic implant indications with just one type of implant and to achieve an improved primary stability between bone and implant contact under different bone conditions. It was completely surprising that the healing risk is reduced by the implant because an ideal primary stability reduces the risk of implant movements during transitional phases from primary stability to secondary stability and then to the final osseointegration.

The implant also has preferably a fine thread, which seamlessly follows the progressive thread. The second screw part in which the fine thread is present may have a cylindrical design. A fine thread in the sense of this invention describes in particular a thread having a smaller thread profile than the thread of the first screw part. It has, in particular, a smaller thread pitch. In the case of a metric thread in particular, the pitch of a thread refers to the thread depth, i.e., the distance between two thread steps along the thread axis, i.e., in other words, the axial distance traveled by one revolution of the thread. It has surprisingly been found that a cylindrical design of the second screw part as a fine thread improves the fusion of the bone and also prevents it from being resorbed, thereby promoting the growth of connective tissue at the cervical end of the implant. The cortical bone reacts strongly to stress peaks in the implant body with remodeling processes, which often result in bone fractures. The progressive multi-start thread of the implant develops specifically into a fine thread, in particular a three-start multi-start thread (also referred to as a three-start thread) in the cervical area of the implant. It was completely surprising that this design creates a larger surface area in the area of the compact bone and that the forces are transferred uniformly from the implant into the bone. This prevents remodeling processes—and the bone remains stable. Compact bone refers in particular to the outer boundary layer of bone (compact bone, substantia compacta), which develops into bone trabeculae and is covered by the periosteum on the outside.

However, the progressive multi-start thread improves the primary stability, which is important in particular for continued loading and/or early loading on the dental implant. The primary stability of an implant is an important criterion for rapid osseointegration and often also for successful subsequent osseointegration. In addition to the conical implant, excellent uniform anchoring in the bone tissue is achieved by the progressive thread. With the progressive thread design, the thickness of the thread increases toward the top. It was completely surprising that the healing process is accelerated by the harmonic, uniform distribution of force, which thus makes possible an immediate load on the implants. Due to the progressive thread, micromovements of the inserted implant are also largely prevented, so that its healing and the integration of the bone are promoted. In contrast with the second screw part, the first screw part has a conical shape, which significantly increases its primary stability, when it is screwed into a straight cavity, in comparison with a cylindrical shape, and thus leads to a perfect adaptation in the cervical area.

With the implants disclosed in the related art, a high primary stability is usually achieved by conical implant bodies, but they result in an unsuitable distribution of force in the various bone structures. In the case of the implant according to the invention, the shapes suitable for the respective bone structure are joined to one another. In this spongy tissue, which can be compressed in a predictable manner, the implant (the first screw part of the implant) is conical, while the implant is cylindrical in the cervical area (in the second screw part). It was completely surprising that the acting forces would be intelligently introduced into the various bone tissues due to the design of the implant and that the bone in situ would be preserved.

This dental implant is preferably rounded at the apical end, which prevents anatomical structures (for example, the sinus bottom, the nasal bottom, the mandibular nerve or the mucous membrane) from being injured when screwing in the implant.

The dental implant has a hexagon socket head on the cervical end, arranged in a conical recess. A superstructure can be connected to the implant by a corresponding retaining screw by means of the hexagon socket head. Such a superstructure and means for fastening same are described in WO 2007/022655 (see also US Patent Publ. 2008/0241792) and WO 2007/022654 (see also US Patent Publ. 2008/0233539), for example.

The superstructure preferably has a connecting part designed as a hexagon head screw, which is also conical and is preferably inserted into the recess with a perfect fit.

The retaining screw, which serves to fasten the superstructure, extends through the superstructure and may thus be screwed into the dental implant. A shoulder on the retaining screw here is preferably supported on a shoulder of the superstructure, so that when the retaining screw is screwed in, the superstructure and the dental implant are tightened and secured.

The superstructure may also be designed with grooves, which permit the use of a snap-on technique for various parts. For example, temporary crowns or impressions can be secured easily by the snap-on technique.

It was completely surprising that a stable connection to the superstructure is established due to the conical recess on the cervical end of the implant in particular. The superstructure can be inserted into the recess easily and without the use of any other tool. Furthermore, cold welding between the implant and the superstructure is achieved in particular due to the conical recess. Cold welding in the sense of the present invention refers in particular to an operation in which primarily metal workpieces of the same material can be joined together at room temperature, so that the connection very closely approximates that of “normal” welding. The basis for cold welding is the fact that the contact surfaces between the implant recess and the outside of the superstructure are in the most extreme limiting case of friction, resulting in a mechanical resistance (friction), which prevents any movement of the contacting parts. Due to the high quality of the material of the implant and the superstructure and the preferably smooth surfaces, a great many metal atoms come in contact at the two interfaces and the forces of attraction even among one another connect them to form a stable atomic lattice.

Two advantages are achieved due to this intimate bond:

First, reducing the risk of a micro-liquid, i.e., the penetration of microbacteria, which would usually otherwise infiltrate into the implant body through micro-cracks and the capillary effects of saliva, and second, [reducing] the risk of micro-movements between the implant superstructure and the implant.

These advantages reduce the risk that bacteria will have a steady supply of nutrients due to a so-called pumping effect (micro-movements). And the bacteria thus permanently secrete toxins in the metabolic process. These toxins cause a strong mouth odor and inflammatory processes in the gingiva and bone and are a partial cause of and/or can trigger peri-implantitis.

The progressive thread advantageously has a separate cylindrical fine thread which develops into a conical thread. A peripheral groove, which can surprisingly prevent the penetration of bacteria and the development of infections, lies at the thread end of the fine thread. The peripheral groove may also be referred to as a thread end reinforcement in the sense of the invention. It was completely surprising that the risk of an intended breaking effect and weakening of the implant can be prevented through reinforcement of the thread end. The thread end reinforcement leads to the end, with a preferred distance from the cervical implant end in the transition to the internal cone, and contributes to the mechanical reinforcement of the implant superstructure stress, making it possible to prevent damage to the implant due to chewing forces acting on the implant, in particular in the case of extra-axial stresses.

In addition, this prevents micro-movements between the implant and the superstructure. On the one hand, this makes it possible to take an accurate impression, while also increasing the precision in subsequent care and treatment, and on the other hand, this ensures long-term stability of the superstructure in the implant. It was completely surprising that due to the peripheral groove in particular, the duration of use of the implant could be increased significantly. Furthermore, the peripheral groove effectively protects the implant and/or the bone tissue surrounding the implant from microbial infections.

It is preferable for the implant to be made of titanium. In the sense of the present invention, titanium is a metallic element from group 4 of the periodic system. An implant is preferably made of cold-worked titanium (in particular pure titanium) of grade 4. It has been found that pure titanium is biologically neutral and does not cause allergic or foreign body reactions. This is due in particular to the fact that titanium enters into a direct molecular bond with the bone, which is not the case with other materials. In addition, it has been found that the bioinert property of titanium and the rapid integration of the implant into the bone can be improved by roughening of the titanium surface. The surface may preferably be etched and/or sandblasted. The surface of the implant is preferably completely blasted with zirconium oxide and acid etched. In experiments with simulated body fluid, the deposition of bioapatite (calcium phosphate) on the implant surface has been proven. This shows the high biocompatibility of the preferred material from the standpoint of a subsequent tight anchoring in the bond.

The surface of the titanium is significantly enlarged by a surface treatment, and optimal osseointegration is achieved by increased deposition of osteogenic cells on the implant. Integration of the implant and/or a homogeneous and stable bond with the surrounding bone can be achieved in the shortest possible amount of time in this way. A surface roughness of 0.5 to 5.0 Ra, preferably 1.0 to 4.0 Ra, especially preferably 1.6 to 3.2 Ra has been found to be particularly preferred. It was completely surprising that the preferred roughness not only improves the integration and/or bonding of the bone tissue to the implant but also makes it possible to apply growth factors or other agents for improving the fusion to the surface. For example, the surface may have a nanostructure onto which biologically active molecules are applied. These may also have an antibacterial effect, so that infections can be prevented. Micro-rough and nano-rough structured implant surfaces are especially preferred because they improve and accelerate the osseointegration of the implant body. An ideal surface geometry for the deposition of the bone cells can be provided through the preferred structuring of the surface. At the same time, the osteoblasts are conducted in settling on the surface (osseoconduction). It is usually even possible for the horizontal shoulder surface of the implant to be surrounded with bone matrix in this way, so that the soft tissue is supported and the predictability of the implant is facilitated.

In the course of the last few years, it has been found that for the predictability of an implantation, it is advisable to take into account the biological width. It is preferable for the biological width of the implant to be 1.9 mm to 2.8 mm. The preferred implant imitates the biological width of the natural alveolar periosteum in subcrestal insertion of the implant. It was completely surprising that an optimal natural protective barrier against bacteria is formed in this way. Furthermore, this also achieves effective protection against peri-implantitis. The biological width of the connective tissue coating at the cervical end of the implant amounts to about 1.5 mm to 3.5 mm in the prior art. The preferred biological width takes into account the biological width of the soft tissue, which entails substantial advantages from an aesthetic and functional standpoint. The biological width in the sense of the invention denotes in particular the distance between the bone boundary and the edge of the implant or of the superstructure.

In a preferred embodiment, it may be advantageous that the dental implant without a superstructure should fuse in place, so that it is advantageous to close its opening. To do so, for example, a locking screw may be used which has a countersunk hexagon head and a thread, which is inserted into the thread of the dental implant. This permits secure closure of the implant to prevent contamination in particular.

It is preferable for the implant to have cutting grooves (e.g., 7) on the apical end (e.g., 2). The implant preferably has at least one, especially two cutting grooves, which serve to receive the bone and secure it against rotation. The cutting grooves preferably serve as relief grooves for the bone shavings. Furthermore, a shortened operating time can be achieved through cutting grooves due to the fact that the use of a thread cutter is eliminated by using a self-cutting double thread, among other things. The implant is easily and conveniently screwed into the final position—nothing further is needed, which greatly simplifies the work of the implantologist.

It is preferable for the recess at the cervical end to have a conicity of 20 to 40 degrees, preferably 25 to 35 degrees and especially preferably 29 to 31 degrees. It has surprisingly been found that an especially stable press-fit between the dental implant and a superstructure can be achieved by a conicity of 20 to 40 degrees. Wobbling movements and damage to the implant or the superstructure can be prevented especially well through an optimal fit of the two elements.

Due to the preferred conicity it is also possible to avoid complex and special connecting elements between the superstructure and the implant. This not only reduces the cost of the implant but also reduces its size, which in turn greatly accelerates the fusion of the surrounding bone tissue. This implant can be used universally for a variety of superstructures due to the conicity and the lack of complex connecting elements. In addition, experiments have shown that the connection is optimally sealed to prevent the penetration of microorganisms by having a conicity of 25 to 35 degrees, which thus makes it possible to prevent infections. Furthermore, a conicity of 29 to 31 degrees is especially advantageous because the implant can be manufactured by a mass production process and there is little waste of material. Due to the preferred conicity, the implant has a material thickness which accelerates the fusion of the implant in the bone because the dimensions of the teeth can be simulated. Furthermore, it is insensitive to the resulting pressures, so that a long service life is ensured. It is also preferable for the recess on the cervical end of the implant to have a conicity of 30 degrees ±0.4 degree (0.01 degree to 10 degrees, preferably 0.2 degree to 8 degrees, especially preferably 0.5 degree to 7 degrees).

The construction of this cone angle is advantageous because it does not have the usual disadvantages for dentists who treat patients with these prosthetic devices in comparison with steeper cone angles because steep conical angles already have a conical self-locking effect even in the early trial stage of implant superstructures in the patient's mouth—as is also the case with impression posts. This results in undesirable jamming of the superstructure parts in the implant, which is unpleasant for the patient and is associated with a very great effort and cost-intensive, time-consuming labor for the treating dentist. These disadvantages can be overcome with the preferred embodiment of the implant.

The preferred conical angles prevent this and are selected so that the conical self-locking in particular effect is achieved only after repositioning the implant superstructure in the patient's mouth and is ultimately achieved only after tightening the prosthetic screw with a predetermined torque in Ncm.

In a preferred embodiment, a chamfered, inwardly directed surface (inwardly directed chamfer, e.g., 11) follows the fine thread toward the cervical end. The fusion of the bone can be improved and its resorption prevented in this way.

In addition, the invention relates to a dental implant kit comprising a dental implant, an impression post, an impression screw and a covering screw. The covering screw has the function of covering the implant during the healing phase. The implant post serves in particular to take an impression of a negative of the oral situation in the mouth and thus allow a precise impression to be made. This can be used in the implant only in conjunction with an implant screw. The impression post can also be used advantageously with divergent-standing implants because of the short hexagon head.

### BRIEF DESCRIPTION OF THE FIGURES

The present invention will now be illustrated below as examples, although it is not limited to them:

FIG. 1 shows a side view of an implant;

FIG. 2 shows a sectional diagram of an implant;

FIG. 3 shows a top view of an implant;

FIG. 4 shows a view of the progressive thread;

FIG. 5 shows a view of the fine thread;

FIG. 6 shows an enlargement of a peripheral groove.

### DESCRIPTION OF VARIOUS AND PREFERRED EMBODIMENTS OF THE INVENTION

All the features in the drawings are disclosed and claimed not only in conjunction with the preferred device illustrated here but are also disclosed as individual features. These disclosed features according to the figures may be both positive and negative features.

Accordingly, each individual feature depicted here (positive or negative feature) in the figures or each feature disclosed in the description of the figures is disclosed as being combinable with other preferred features of the description of the claims.

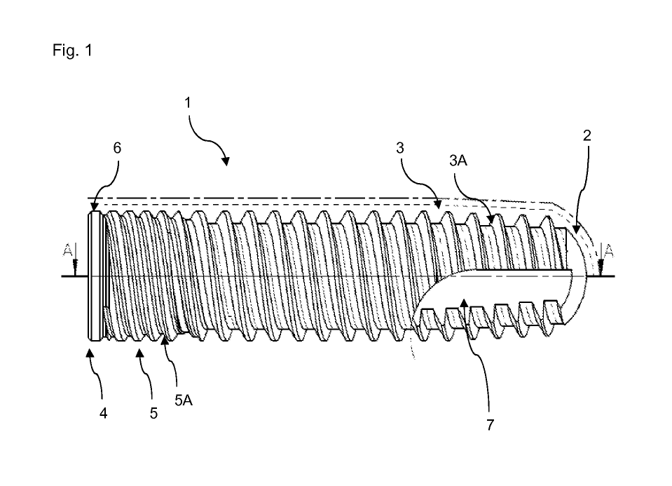

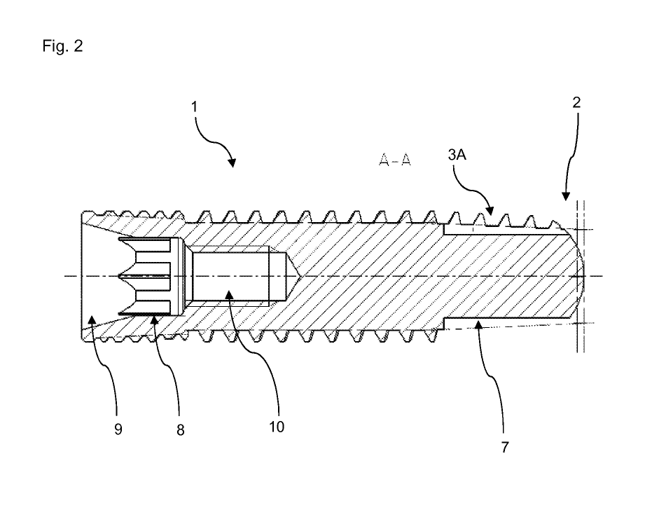

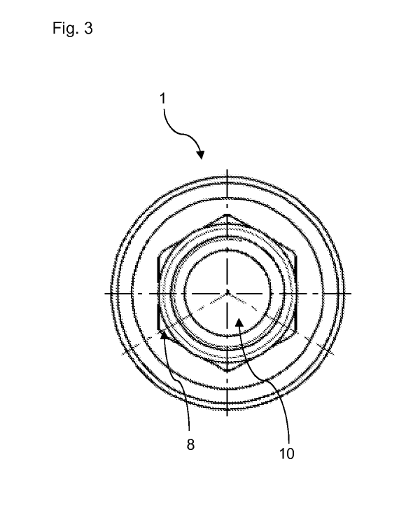

FIG. 1 shows a side view of an implant, FIG. 2 shows a sectional diagram of an implant, and FIG. 3 shows a view of an implant from above. The implant 1 is preferably made of pure titanium (e.g., cold-worked titanium of grade 4) and has a rounded apical end 2. The first screw part 3 with the progressive thread 3A, which is arranged on the apical end 2, is followed by a second screw part 5 with a fine thread 5A (also referred to as a micro-thread). The first screw part 3 has a minimal conicity in particular and the second screw part 5 is designed to be cylindrical. In this exemplary embodiment, the progressive thread 3A is a two-start self-cutting thread, which becomes wider preferably from the apical end 2 to the other cervical end 4, followed by a fine thread 5A (e.g., a triple-start multi-thread). Toward the cervical end 4, the fine thread 5A is connected to a peripheral groove 6. The first screw part 3 also has a conical shape, which tapers toward the apical end 2, so that the primary stability of the implant 1 is increased. In addition, the connection to the jawbone into which the implant 1 is introduced is improved by the cylindrical shape of the second screw part 5. The implant also has cutting grooves 7 in the apical area 2, for example, two grooves which also serve as relief grooves for the resulting bone shavings. FIG. 2 also shows that the implant has a hexagon socket head 8 on the cervical end 4 arranged in a conical recess 9 with a bore 10 preferably having a thread connected to it. This means that the implant preferably has a bore 10 in particular with a thread. Simple fastening of a retaining screw or of a superstructure is possible due to the conical recess 9 in particular.

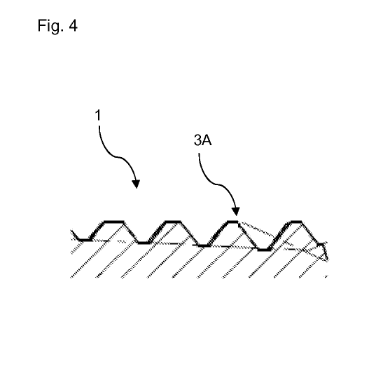

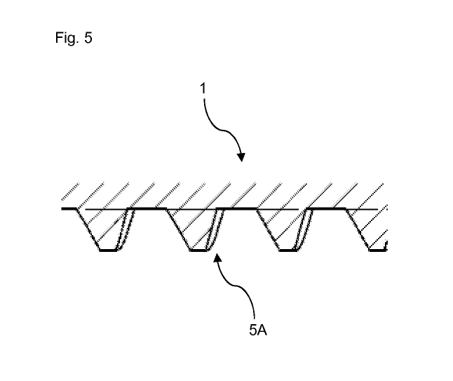

FIGS. 4 and 5 show a detail of the thread of the first and second screw parts. The first screw part having a conical design has a progressive thread 3A, which may be a triple-start multi-thread, for example. However, the thread in the second screw part, which is connected to the progressive thread 3A, is a fine thread 5A which may also be a two-start thread.

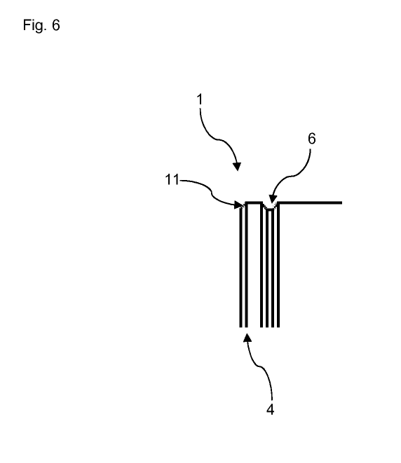

FIG. 6 shows an enlargement of the peripheral groove. A peripheral groove 6, which surprisingly prevents the penetration of bacteria into the bore in the jawbone, is situated on the cervical end 4 of the implant 1. It may be advantageous if a chamfer 11 directed inward is connected to the fine thread 5A toward the cervical end 4. However, fusion of the implant 1 can be greatly accelerated, and dangerous infections can be prevented by the groove also due to this chamfer.

It will be appreciated that the implant and kit of the present invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive. The figures illustrate a preferred embodiment of the invention, and the features illustrated there may also be combined with other embodiments of the invention.

### LIST OF REFERENCE NUMERALS

1 Implant

2 Apical end

3 First screw part

3A Progressive thread

4 Cervical end

5 Second screw part

5A Fine thread

6 Peripheral groove/thread end reinforcement

7 Cutting grooves

8 Hexagon socket head

9 Recess

10 Bore

11 Chamfer

1. A dental implant comprising
a first screw part, which is conical, and
a second screw part, which is cylindrical,
the first screw part comprising a progressive thread, which becomes progressively wider starting from a rounded apical end, and the second screw part comprising a fine thread connected to the progressive thread, wherein there is a peripheral groove at a thread end of the fine thread and wherein the implant has a hexagon socket head on the cervical end arranged in a conical recess.

2. The dental implant according to claim 1, wherein the implant is made of titanium.

3. The dental implant according to claim 1, wherein the surface of the implant is etched and/or sandblasted.

4. The dental implant according to claim 1, wherein the roughness of the surface amounts to 0.5 to 4.0 Ra.

5. The dental implant according to claim 1, wherein the biological width of the implant is 1.9 mm to 2.8 mm.

6. The dental implant according to claim 1, wherein the implant has cutting grooves on the apical end.

7. The dental implant according to claim 1, wherein the recess on the cervical end has a conicity of 20 to 40 degrees.

8. The dental implant according to claim 1, wherein an inwardly directed chamfer follows the fine thread toward the cervical end.

9. The dental implant according to claim 1, wherein the implant has a bore with a thread.

10. The dental implant kit, comprising a dental implant according to claim 1, an impression post, an impression screw and a covering screw.

11. The dental implant according to claim 4, wherein the roughness of the surface amounts to 1.0 to 4.0 Ra.

12. The dental implant according to claim 11, wherein the roughness of the surface amounts to 1.6 to 3.2 Ra.

13. The dental implant according to claim 7, wherein the recess on the cervical end has a conicity of 25 to 35 degrees.

14. The dental implant according to claim 7, wherein the recess on the cervical end has a conicity of 29 to 31 degrees.
